(19) United States
(12) Plant Patent Application Publication
(10) Pub. No.: US 2006/0162028 P1
(43) Pub. Date: Jul. 20, 2006

(54) Mushroom named "Hokuto NT-100"

(76) Inventors: Hitomi Naganuma, Nagano-shi (JP); Eriko Tominaga, Nagano-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 11/004,584

(22) Filed: Dec. 6, 2004

(51) Int. Cl. A01H 15/00

(52) U.S. Cl. 800/297

(57) ABSTRACT

A new and distinct variety of mushroom, named Hokuto NT-100, is described, which mushroom has the following characteristics: large cap, thick meat, nice consistency, nice taste, uniform growth when cultivated in a facility, and suitable for bottle cultivation. The variety of mushroom named Hokuto NT-100 was developed using Hokuto MY-95 and Hokuto MY-75, which are registered varieties of Hokuto Corporation, of Nagano-shi Japan, and a wild variety found in Aomori Prefecture of Japan.

### BACKGROUND OF THE INVENTION

The present invention relates to a new and distinct variety of mushroom having the following characteristics: large cap, thick meat, nice consistency, nice taste, uniform growth when cultivated in a facility, and suitable for bottle cultivation. The new variety of mushroom named Hokuto NT-100 was developed using Hokuto MY-95 and Hokuto MY-75, which are registered varieties of Hokuto Corporation, of Nagano-shi Japan, and a wild variety found in Aomori Prefecture of Japan.

### BRIEF DESCRIPTION OF THE DRAWINGS

FIG. 1 is a top view of the Hokuto NT-100 mushroom;

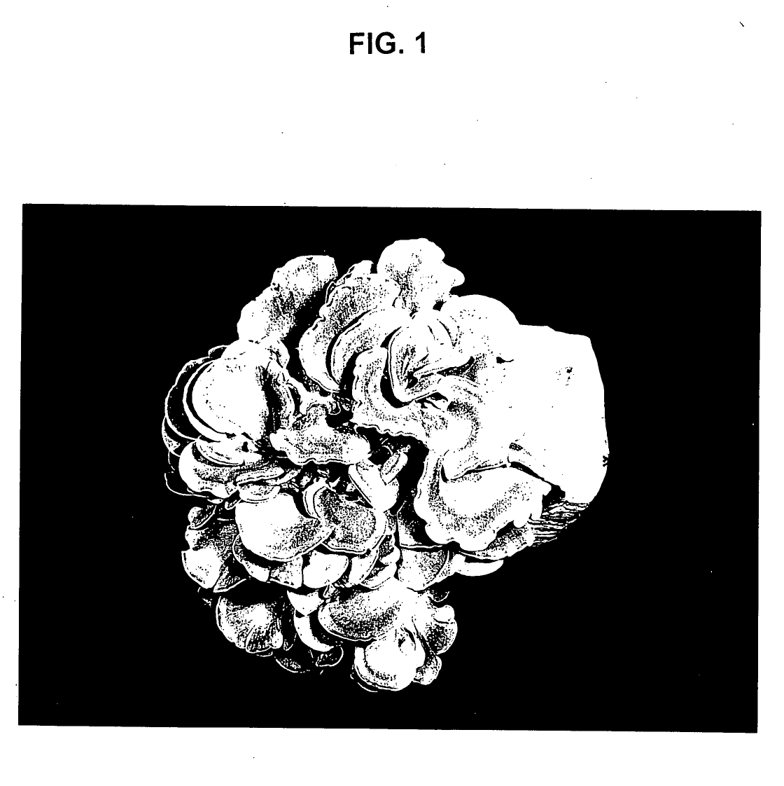

FIG. 2 is an oblique view of the Hokuto NT-100 mushroom;

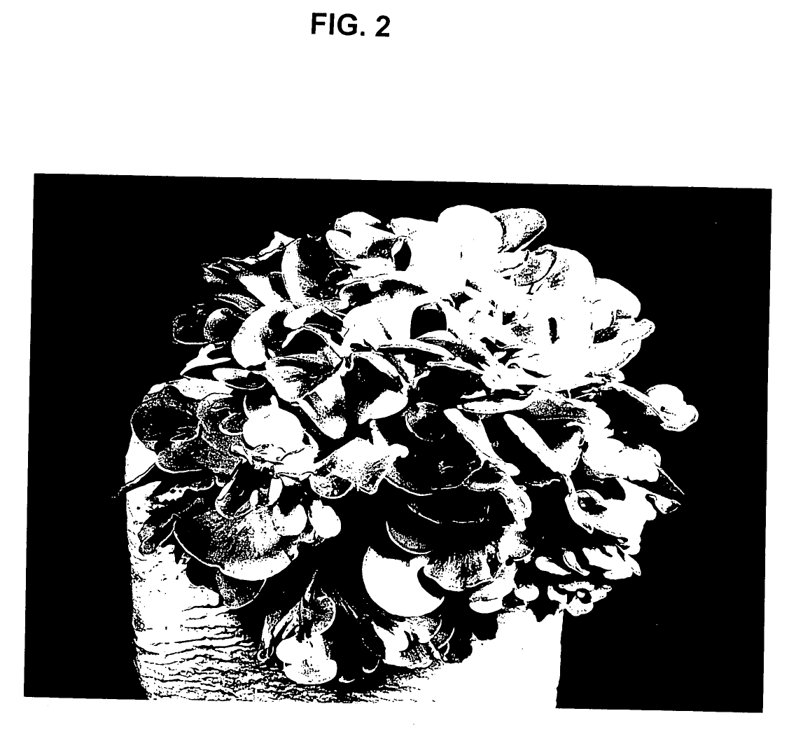

FIG. 3 is a partial side view of the Hokuto NT-100 mushroom;

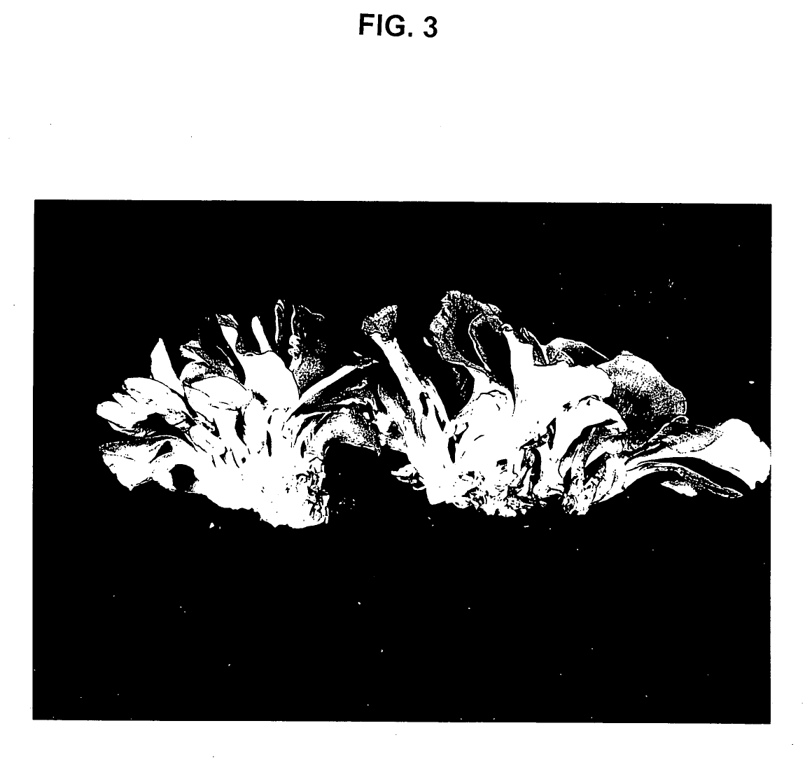

FIG. 4 is an oblique view of the Hokuto NT-100 mushroom in bottle cultivation;

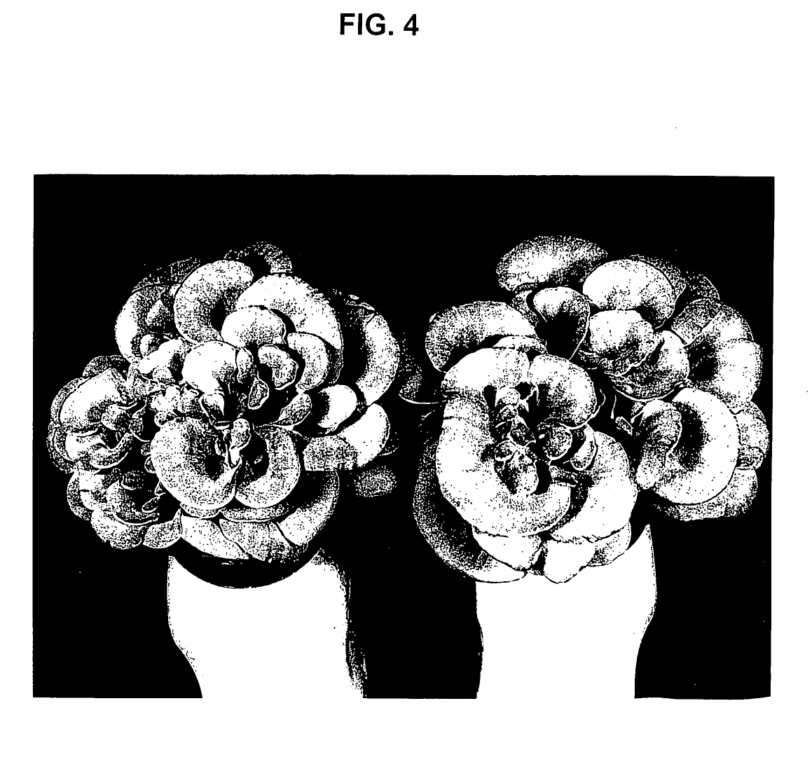

FIG. 5 is a side view of the Hokuto NT-100 mushroom in bottle cultivation;

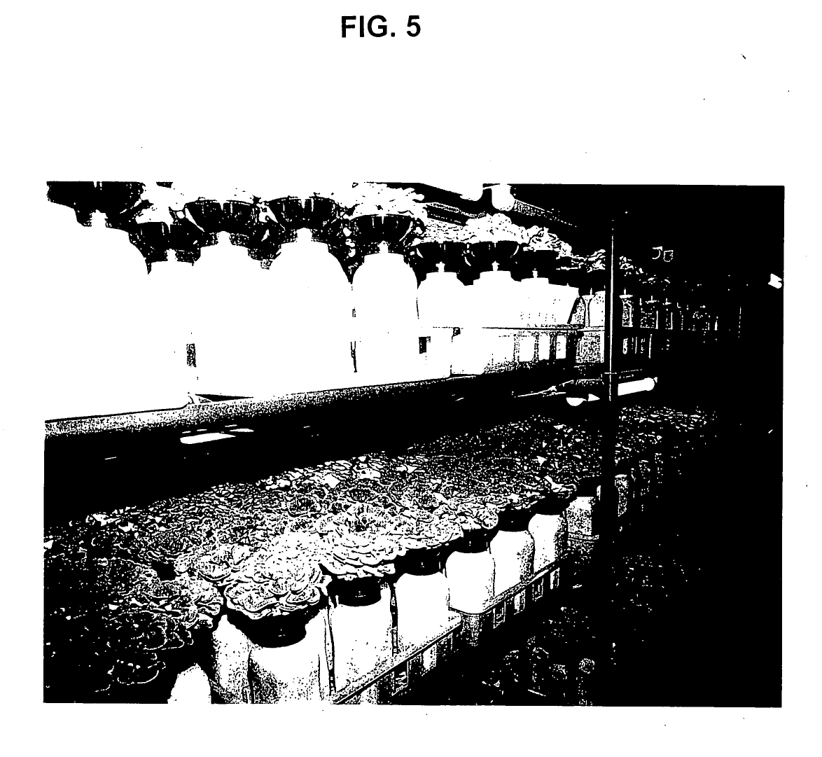

FIG. 6 is a top view of the Hokuto NT-100 mushroom in outdoor cultivation;

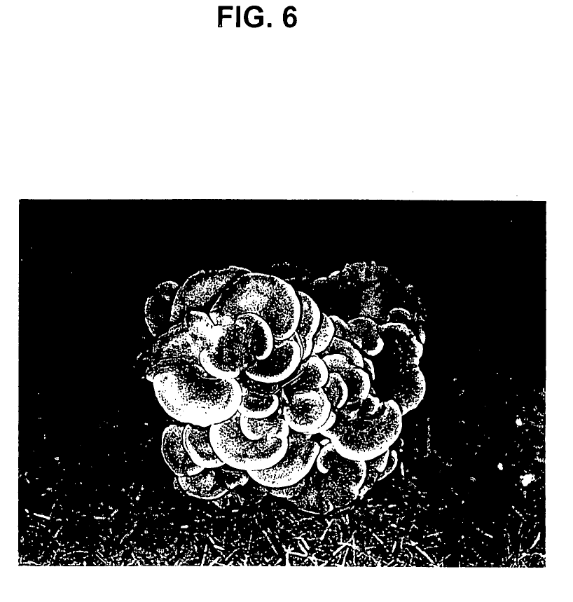

FIG. 7 is a top view of the Hokuto MY-95 mushroom for the purposes of comparison;

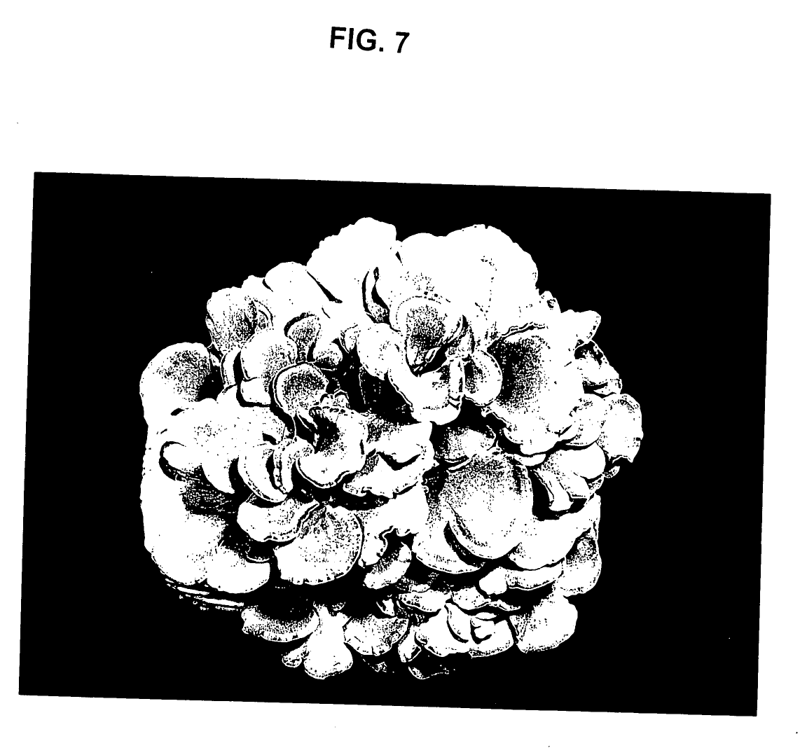

FIG. 8 is a side view of the Hokuto MY-95 mushroom;

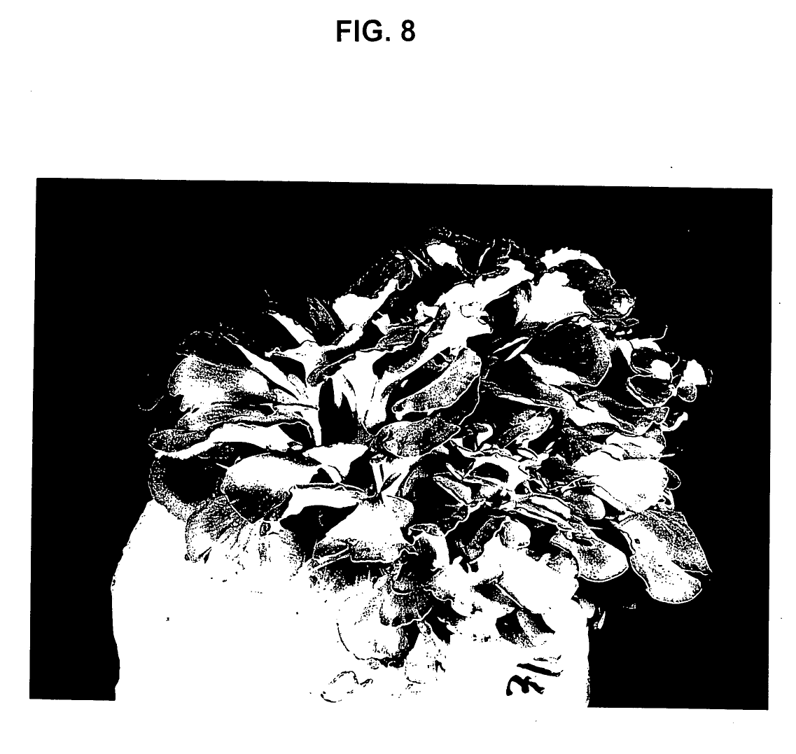

FIG. 9 is a top view of the Hokuto MY-95 mushroom in outdoor cultivation;

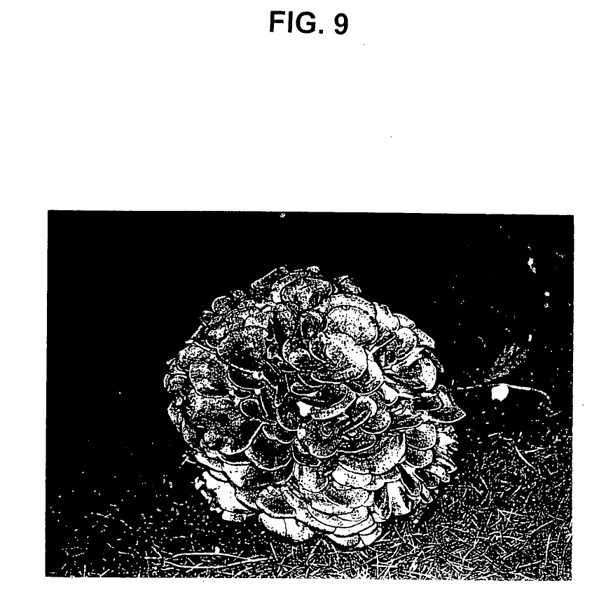

FIG. 10 is a top view of the MH18211 mushroom for the purposes of comparison;

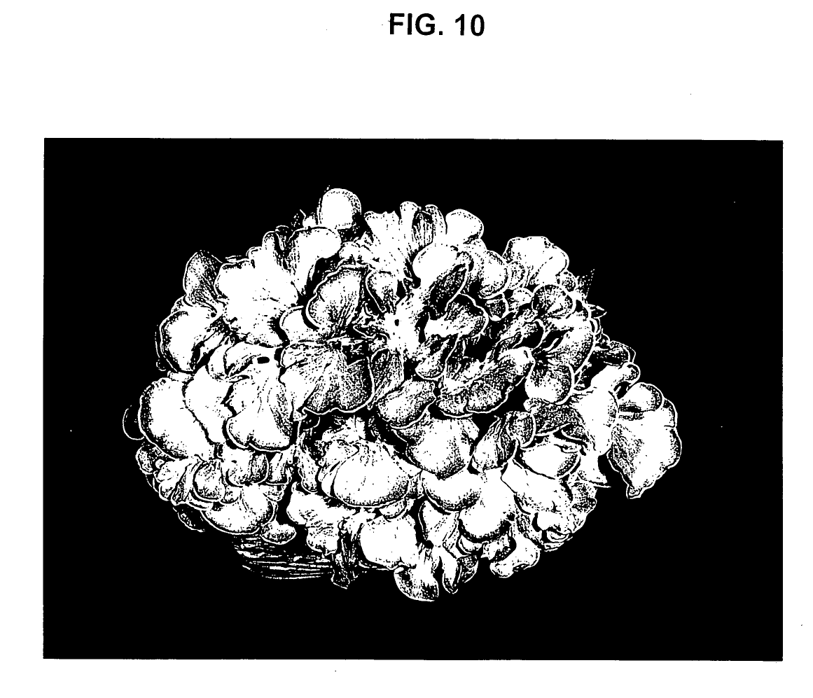

FIG. 11 is a side view of the MH18211 mushroom; and

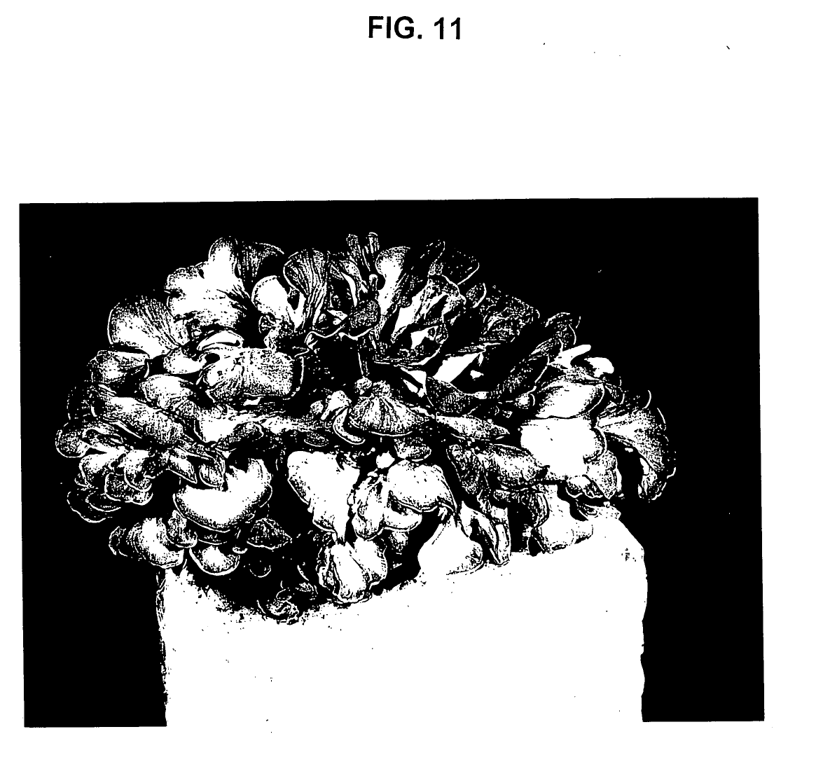

FIG. 12 is a top view of the MH18211 mushroom in outdoor cultivation.

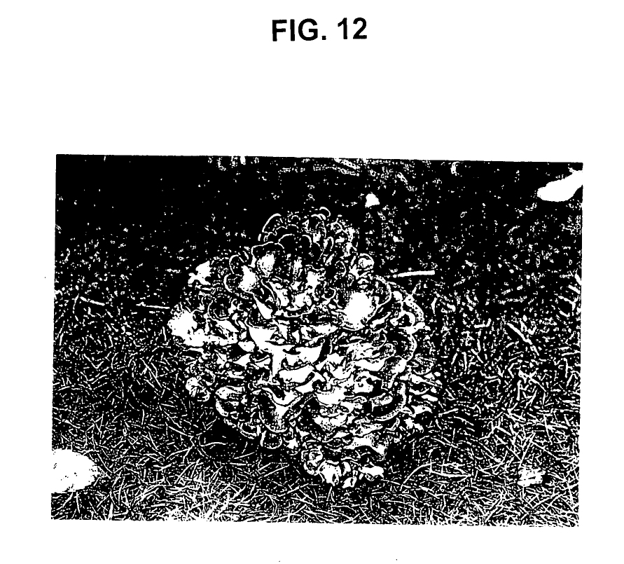

### DETAILED DESCRIPTION OF THE INVENTION

The history of the Hokuto NT-100 mushroom in terms of improvement period and the like are set forth in the following chronological list of each stage of variety improvement:

January 1993: By crossing MH18206 and MH18215, the following three fungal strains were obtained: Hokuto MY-75, Hokuto MY-95 and GD99.

February 1995: Hyphae were obtained from Hokuto MY-75 and GD99.

March 1995: By crossing the hyphae of Hokuto MY-75 and GD99, MH182114 was obtained.

May 1996: From Hokuto MY-95, hyphae were obtained.

June 2000: From MY19284, hyphae were obtained.

August 2000: By crossing the hyphae of MH18284 and MY-95, MH182113 was obtained.

November 2000: From MH182114, hyphae were obtained.

July 2001: From MH182113, hyphae were obtained

January 2002: By crossing the hyphae of MH182113 and MH182114, a superior variety MH182115 was obtained.

March 2002 to March 2003: Tests were repeated to identify the characteristics of MH182115, and during this time, it was found that bottle cultivation was favorable, and stability, uniformity and differentiability were confirmed. In November 2002, MH182115 was named Hokuto NT-100 and variety improvement was completed. More tests were conducted after that, and characteristics tests were completed in March 2003.

The system which resulted in the Hokuto NT-100 mushroom can be summarized as follows:

Crossbreeding System 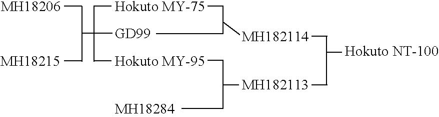
The Hokuto NT-100 mushroom has the following characteristics: large cap, thick meat, nice consistency, nice taste, uniform growth when cultivated in a facility, and suitable for bottle cultivation.

When Hokuto NT-100 was cultured with commercially available varieties, inhibition zones were seen. In addition, the optimal temperature for hyphal growth was approximately 24 C.

The morphological characteristics of Hokuto NT-100 are as follows: at the time of harvest, the cap is fan-shaped or circular; the margin is rounded off; hair is minimal; pore shape is elliptical (Type 1); and pore cross section is uneven. When naturally cultivated, the mode of development is medium and development occurs in September. Experimental data have also shown that the length of time from inoculation to development is 63 days; the optimal development temperature is 18-20 C; the optimal growth temperature is 17-18 C; and yield is 547.2 g.

With regard to other characteristics, the vitamin D₂ content of Hokuto NT-100 was high at 20.6 mg/100 g (international units: 824 IU) according to experimental data.

Some of these characteristic tests for Hokuto NT-100 as compared to similar mushroom varieties Hokuto MY-95 and MH18211 (Mori Sangyo #51) are summarized in the following table:

| | Characteristic code of filed variety | | | | |
|---|---|---|---|---|---|
| Characteristics | 01 | 02 | 03 | 04 | 05 |
| Genetic | | | | | |
| Dual culture | | | | | |
| Dislike-touch reaction | − | | | | |
| Physiology | | | | | |
| Mycelial growth | | | | | |
| Effect of temperature | | | | | |
| The optimum (° C.) | 22 | 23 | \textcircled{24} | 25 | 26 |
| Growth rate | | | | | |
| 10° C. | | | | | |
| 15° C. | | | | | |
| 20° C. | | | | | |
| 25° C. | | | | | |
| 30° C. | | | | | |
| 35° C. | | | | | |
| Mycelial color | [symbol] | | | | |
| shape of the surface of | [symbol] | taugh | | | |
| the colony | | | | | |
| Shape of colony edge | [symbol] | | | | |
| colony thickness | | | thin | | medium |
| Density of hyphae | | | [symbol] | | medium |
| Cultivation | | | | | |
| characteristics | | | | | |
| Capsule formation | | | little | | [symbol] |
| Capsule coloration | [symbol] | | | | |
| Length of time until | <30 | 31˜ | 41˜ | 51˜ | [symbol] |
| fruit body fromation at | | 40 | 50 | 60 | |
| optimal temperature | | | | | |
| Development period | | | | | |
| (natural cultivation) | | | | | |
| Optimal development | <15 | [symbol] | 20˜ | >25 | |
| temperature (° C.) | | | 25 | | |
| Optimal fruit body | [symbol] | 18˜ | >21 | | |
| growth temperature | | 21 | | | |
| (C.) | | | | | |
| Other | | | | | |
| Budding period | | | Early | | [symbol] |
| (natural cultivation) | | | | | |
| Yield (g/bottle) | | | | | |
| Morphology | | | | | |
| Cap | | | | | |
| Size | | | small | | medium |
| Thickness | | | Thin | | [symbol] |
| Notch | [symbol] | | | | |
| Cross sectional shape | 1 | \textcircled{2} | 3 | | |
| Face color | \textcircled{W} | G-W | D- | B | D-B |
| | | | G | | |
| Annulus | 1 | 2 | \textcircled{3} | 4 | |
| Hair | | | [symbol] | | medium |
| Tube | | | | | |
| Shape of tube face | \textcircled{1} | 2 | 3 | 4 | |
| Uneveness of cross | Absent | | | | |
| sectional the tip of | | | | | |
| tube | | | | | |
| Tube position | 1 | 2 | 3 | | |
| Stipe shape | 1 | \textcircled{2} | 3 | | |

| | Characteristic code of | | | | |
|---|---|---|---|---|---|
| | filed variety | | | | Note |
| Characteristics | 06 | 07 | 08 | 09 | (Data) |
| Genetic | | | | | |
| Dual culture | | | | | |
| Dislike-touch reaction | | | | + | − |
| Physiology | | | | | |
| Mycelial growth | | | | | |
| Effect of temperature | | | | | |
| The optimum (° C.) | 27 | 28 | 29 | 30 | 24° C. |
| Growth rate | | | | | |
| 10° C. | | | | | 0.8 |
| 15° C. | | | | | 1.7 |
| 20° C. | | | | | 2.4 |
| 25° C. | | | | | 2.6 |
| 30° C. | | | | | 1.0 |
| 35° C. | | | | | 0.1 |
| Mycelial color | | | | pther | white |
| shape of the surface of | | | | other | smaooth |
| the colony | | | | | |
| Shape of colony edge | | | | regular | irregular |
| colony thickness | | [symbol] | | | thick |
| Density of hyphae | | dense | | | sparse |
| Cultivation | | | | | |
| characteristics | | | | | |
| Capsule formation | | much | | | medium |
| Capsule coloration | | | | present | absent |
| Length of time until | >70 | | | | 63 days |
| fruit body fromation at | | | | | |
| optimal temperature | | | | | |
| Development period | | | | | 6 month |
| (natural cultivation) | | | | | |
| Optimal development | | | | | 18˜20° C. |
| temperature (C.) | | | | | |
| Optimal fruit body | | | | | 17˜18° C. |
| growth temperature | | | | | |
| (C.) | | | | | |
| Other | | | | | |
| Budding period | | Late | | | intermediate |
| (natural cultivation) | | | | | |
| Yield (g/bottle) | | | | | 547.2 g |
| Morphology | | | | | |
| Cap | | | | | |
| Size | | [symbol] | | | 56.4 mm |
| Thickness | | Thick | | | 2.6 mm |
| Notch | | | | present | Absent |
| Cross sectional shape | | | | | type 2 |
| Face color | | | | other | dark Brown |
| Annulus | | | | other | type 3 |
| Hair | | many | | | few |
| Tube | | | | | |
| Shape of tube face | | | | other | type 1 |
| Uneveness of cross | | | | [symbol] | present |
| sectional the tip of | | | | | |
| tube | | | | | |
| Tube position | | | | other | type 3 |
| Stipe shape | | | | other | type 2 |

| | Code of the | |
|---|---|---|
| | similar variety | |
| Characteristics | Hokuto MY- | |
| 95 | HM18211 | |
| Genetic | | |
| Dual culture | | |
| Dislike-touch reaction | 09 | 09 |
| Physiology | | |
| Mycelial growth | | |
| Effect of temperature | | |
| The optimum (° C.) | 04 | 05 |
| Growth rate | | |
| 10° C. | 0.9 | 0.8 |
| 15° C. | 1.4 | 1.8 |
| 20° C. | 1.6 | 2.5 |
| 25° C. | 2.0 | 2.9 |
| 30° C. | 1.2 | 2.7 |
| 35° C. | 0.1 | 0.2 |
| Mycelial color | 01 | 01 |
| shape of the surface of | 01 | 01 |
| the colony | | |
| Shape of colony edge | 01 | 09 |
| colony thickness | 05 | 03 |
| Density of hyphae | 03 | 03 |
| Cultivation | | |
| characteristics | | |
| Capsule formation | 05 | 05 |
| Capsule coloration | 01 | 09 |
| Length off time until | 05 | 06 |
| fruit body fromation at | | |
| optimal temperature | | |
| Development period | 6 month | 7 month |
| (natural cultivation) | | |
| Optimal development | 022 | 02 |
| temperature (C.) | | |
| Optimal fruit body | 01 | 01 |
| growth temperature | | |
| (C.) | | |
| Other | | |
| Budding period | 05 | 05 |
| (natural cultivation) | | |
| Yield (g/bottle) | 575.9 g | 338.9 g |
| Morphology | | |
| Cap | | |
| Size | 07 | 07 |
| Thickness | 05 | 05 |
| Notch | 01 | 09 |
| Cross sectional shape | 02 | 02 |
| Face color | Grayish | Grayish |
| | brown | yellow |
| Annulus | 03 | 02 |
| Hair | 03 | 03 |
| Tube | | |
| Shape of tube face | 02 | 01 |
| Uneveness of cross | 09 | 09 |
| sectional the tip of | | |
| tube | | |
| Tube position | 03 | 03 |
| Stipe shape | 02 | 01 |

Distinguishable characteristics of Hokuto NT-100 are set forth in the following:
When cultured with Hokuto MY-95, an inhibition zone is seen. Hokuto NT-100 is different from Hokuto MY-95 in the following characteristics: optimal growth temperature is 24 and 26 C, respectively; meat thickness is thick and regular, respectively; yield is 547.2 and 575.9 g, respectively; cap surface color is dark red and grayish brown, respectively; and pore surface shape is type 1 and 2, respectively. When cultured with MH18211, an inhibition zone is seen. Hokuto NT-100 is different from MH18211 in the following characteristics: optimal growth temperature is 24 and 28 C, respectively; margin shape is irregular and regular, respectively; meat thickness is thick and regular, respectively; bottom capsule is present and absent, respectively; length of time from inoculation to development is 63 and 73 days, respectively; length of time for natural development is 6 and 7 months, respectively; yield is 547.2 and 338.9 g, respectively; cap gill is present and absent, respectively; cap surface is color dark red and grayish yellow, respectively; annellation type is 3 and 2, respectively; and cross section shape of stipe is type 2 and 1, respectively.

The above comparative test results were based, at least in part, on the following tests where the cultivation conditions under which the above-mentioned characteristic tests were conducted were as follows:

a. Cultivation site: Mushroom Research Laboratory, Hokuto Corporation, Nagano, Japan.

b. Cultivation period: March 2002 to March 2003

c. Cultivation methods (location, facility, medium, cultivation mode, scale, etc.). Mushrooms listed in the test results were cultivated using conventional methods in a culture room with 300 containers and a growth room with 300 containers. In the characteristics tests, cultivation was performed using ten bags per block and the cultivation experiment was repeated twice.

The following tests were conducted:

(1) Dual Culture—conducted according to the examination standards for mushrooms where characteristics were observed on Difco PDA medium. The strains used were: MH18211 Commercial strain (Mori Sangyo #51)

MH182113 mating strain

MH182114 mating strain

MH18284 Wild type

Hokuto MY-75

Hokuto MY-95

MH18212 Commercial strain (Fujimoto sangyou M10)

MH18222 Commercial strain (Tochiokimaitake)

MH148261 Commercial strain (Tokatiana)

With regard to the dislike-touch reaction, the results were as follows where + is present and − is absent.

| | Hokuto NT-100 |
|---|---|
| Hokuto NT-100 | − |
| MH18211 | + |
| MH182113 | + |
| MH182114 | + |
| MH18284 | + |
| Hokuto MY-75 | + |
| Hokuto MY-95 | + |
| MH18212 | + |
| MH18222 | + |
| MH148261 | + |

(2) Mycelial growth—the growth of Hokuto NT-100 was compared with Hokuto MY-95 and MH18211.

temperature(° C.)

| | 10 | 15 | 20 | 25 | 30 | 35 |
|---|---|---|---|---|---|---|
| Hokuto NT-100(mm/day | 0.8 | 1.7 | 2.4 | 2.6 | 1.0 | 0.1 |
| Hokuto MY-95(mm-day) | 0.9 | 1.4 | 1.6 | 2.0 | 1.2 | 0.1 |
| MH18211(mm/day) | 0.8 | 1.8 | 2.5 | 2.9 | 2.7 | 0.2 |

| | temperature(° C.) | | | | | | |
|---|---|---|---|---|---|---|---|
| | 20 | 22 | 24 | 26 | 28 | 30 | optimum temp. |
| Hokuto NT-100(mm/day | 2.3 | 2.5 | 2.6 | 2.5 | 2.1 | 1.3 | 24 |
| Hokuto MY-95(mm-day) | 1.6 | 1.8 | 2.1 | 2.2 | 2.0 | 1.9 | 26 |
| MH18211(mm/day) | 2.4 | 2.8 | 3.0 | 3.2 | 3.3 | 3.2 | 28 |

(3) Characteristics data—cultivation data for Hokuto NT-100 was compared with Hokuto MY-95 and MH18211.

| Characteristics | Hokuto NT-100 | Hokuto MY-95 | MH18211 |
|---|---|---|---|
| Capsule | Medium | Medium | Medium |
| formation | | | |
| Capsule | Absent | Absent | Present |
| coloration | | | |
| Length of time | 60-63 days | 60-63 days | 71-73 days |
| until fruit body | | | |
| formation at | | | |
| optimal temper- | | | |
| ature | | | |
| Optimal develop- | 18—21 C. | 18-21 C. | 18-20 C. |
| ment temperature | | | |
| Optimal fruit | 17-18 C. | 17-18 C. | 17-18 C. |
| body growth | | | |
| temperature | | | |
| Development | September | September | September |
| period (nat- | | | |
| ural cultivation) | | | |
| Yield | 574.2 ± 2.6 g | 575.9 ± 53.8 g | 338.9 ± 44.7 g |
| Cap size | 56.4 ± 16.5 mm | 43.6 ± 15.8 mm | 45.1 ± 10.9 |
| | | | mm |
| Cap thickness | 2.6 ± 0.4 mm | 2.6 ± 0.5 mm | 2.6 ± 0.5 mm |
| Cap surface color | Dark yellow red | Grayish brown | Grayish |
| | (1910) | (1919) | yellow |
| | | | (2212) |

(4) Temperature data—data for the optimum development temperature of Hokuto NT-100 was determined using conventional cultivation methods where fungal development was monitored at different temperatures, the results being as follows:

TABLE 1
Primordium formation at each development temperature

| Development | |
|---|---|
| temperature (C.) | Hokuto NT-100 |
| <15 | Primordium formation occurred 61 days |
| | after inoculation |
| 16-20 | Primordium formation occurred 56 days |
| | after inoculation |
| 21-25 | Low pigmentation on the culture |
| | medium surface |
| >25 | No budding |

As shown in Table 1, while primordium formation occurred at temperatures below 20° C., when considering cultivation style and productivity, earlier primordium formation is desirable. Therefore temperatures between 16 and 20° C. were suitable, and, in this temperature range, development was most favorable at 18-20° C.

(4) Vitamin D2 Content

The vitamin D2 content in the fruit body was measured with HPLC and the results were as follows:

| | Hokuto | Hokuto | |
|---|---|---|---|
| | NT-100 | MY-95 | HM18211 |
| The vitamine D2 | 20.6 | 15.6 | 7.1 |
| (μg/100 g) | | | |
| The vitamine D2 | 824.0 | 624.0 | 280.0 |
| (IU/100 g) | | | |

What is claimed as new and is desired to be secured by Letters Patent of the United States is:

1. A new and distinct variety of mushroom, substantially as described and claimed.
